(19) United States
(12) Patent Application Publication
(10) Pub. No.: US 2025/0120579 A1
(43) Pub. Date: Apr. 17, 2025

(54) MEDICAL DEVICES WITH ILLUMINATION DEVICES AND RELATED SYSTEMS AND METHODS OF USE

(71) Applicant: Boston Scientific Scimed, Inc., Maple Grove, MN (US)

(72) Inventors: Louis J. BARBATO, Franklin, MA (US); Kirsten VIERING, Newton, MA (US)

(73) Assignee: Boston Scientific Scimed, Inc., Maple Grove, MN (US)

(21) Appl. No.: 18/912,212

(22) Filed: Oct. 10, 2024

Related U.S. Application Data
(60) Provisional application No. 63/589,385, filed on Oct. 11, 2023.

(51) Int. Cl. A61B 1/05; A61B 1/00; A61B 1/06

(52) U.S. Cl. CPC A61B 1/05; A61B 1/00096; A61B 1/0661

(57) ABSTRACT

According to one aspect, a medical device may comprise a shaft extending to a distal portion. The distal portion of the shaft may include a first imaging device; an electrical cable coupled to the first imaging device and extending proximally from the first imaging device; and a first illumination device positioned entirely proximal from the first imaging device. The first illumination device is (i) coupled to the electrical cable and (ii) configured to illuminate a first field of view of the first imaging device.

### CROSS-REFERENCE TO RELATED APPLICATIONS

This application claims the benefit of priority to U.S. Provisional Application No. 63/589,385, filed on Oct. 11, 2023, which is incorporated by reference herein in its entirety.

### TECHNICAL FIELD

Various aspects of this disclosure relate generally to distal tips of medical devices, systems, and related methods. In particular, aspects of this disclosure relate to illumination features of scope devices, among other aspects.

### BACKGROUND

Endoscopes have attained great acceptance within the medical community since they provide a means for performing procedures with minimal patient trauma while enabling the physician to view the internal anatomy of the patient. Over the years, numerous endoscopes have been developed and categorized according to specific applications, such as cystoscopy, colonoscopy, laparoscopy, upper GI endoscopy and others. Endoscopes may be inserted into the body's natural orifices or through an incision in the skin. An endoscope is usually an elongated tubular shaft, rigid or flexible, having a video camera or a fiber optic lens assembly at its distal end. The shaft is connected to a handle, and viewing is usually possible via an external screen. Various surgical tools may be inserted through a working channel in the endoscope for performing different surgical procedures. Endoscopes, such as colonoscopes, that are currently being used typically have a front camera for viewing the internal organ, such as the colon, an illuminator, a fluid injector for cleaning the camera lens and sometimes also the illuminator, and a working channel for insertion of surgical tools, for example, for removing polyps found in the colon. Often, endoscopes also have fluid injectors (“jet”) for cleaning a body cavity, such as the colon, into which they are inserted. The illuminators commonly used are light-emitting diodes (LEDs) for illumination. Among the disadvantages of such endoscopes are their limited space at the distal tip for operating medical and surgical tools, for example, due to the LEDs and camera positioned within the distal tip. Also, the size of illuminators may be limited due to their position within the distal tip.

The current disclosure may solve one or more of these issues or other issues in the art.

### SUMMARY

Embodiments of the this disclosure relate to, among other things, medical devices and systems for visualizing internal patient anatomy, and related methods of use. Embodiments of this disclosure may provide space efficient component configurations within the tip section of a medical device, and/or may help to enable efficient packing of necessary elements in the tip section, while maintaining their functionality. Each of the embodiments disclosed herein may include one or more of the features described in connection with any of the other disclosed embodiments.

According to one aspect, a medical device may comprise a shaft extending to a distal portion. The distal portion of the shaft may include a first imaging device; an electrical cable coupled to the first imaging device and extending proximally from the first imaging device; and a first illumination device positioned entirely proximal from the first imaging device. The first illumination device is (i) coupled to the electrical cable and (ii) configured to illuminate a first field of view of the first imaging device.

In other aspects, the medical device may include one or more of the following features. The distal portion may include a first window at a distal front face of the shaft, and the first window may be longitudinally aligned with the first imaging device and the first illumination device. A second illumination device may be positioned entirely proximal from the first imaging device, and the second illumination device may be (i) coupled to the electrical cable and (ii) configured to illuminate the first field of view of the first imaging device. A distal front face of the distal portion may be entirely transparent. At least one sidewall of the distal portion may be transparent. The medical device may further include a second imaging device, and the first illumination device may be entirely proximal from the second imaging device. A first field of view of the first imaging device may face a distal direction, a second field of view of the second imaging device may face a direction transverse from a central longitudinal axis of the distal portion, and the first illumination device may be configured to illuminate both the first field of view and the second field of view.

In other aspects, the medical device may include one or more of the following features. A sidewall of the distal portion may include a window aligned with the second imaging device. The distal portion may further include an optical fiber coupled to the first illumination device and extending distally to a distal front face of the distal portion. The distal portion may further comprise at least one transparent portion comprising phosphor, and the at least one transparent portion may be configured to convert blue light to white light. The distal portion may further comprise a prism configured to redirect light received from the first illumination device towards a field of view of the first imaging device. The distal portion may further comprise a first optical fiber coupled to the prism. The distal portion may further comprise a second optical fiber coupling the prism to the first illumination device. The first illuminator may include a first illumination surface, and the first illumination surface may extend transverse from a central longitudinal axis of the distal portion. The distal portion may further comprise a second imaging device, and the field of illumination of the first illumination device may also be directed towards a second field of view of the second imaging device.

In other aspects, a medical device may comprise a shaft extending to a distal portion, the distal portion of the shaft including: a front-facing imaging device; a side-facing imaging device; an electrical cable coupled to the front-facing imaging device and the side-facing imaging device; and a first illumination device positioned entirely proximal from the front-facing imaging device and the side-facing imaging device. The first illumination device may be configured to illuminate a first field of view of the first imaging device and a second field of view of the second imaging device.

In other aspects, the medical device may include one or more of the following features. The distal portion may further include a distal cap, and the distal cap may contain the front-facing imaging device, the side-facing imaging device, and the first illumination device; and the distal cap may be at least partially transparent. A radially-inward facing surface, relative to a central longitudinal axis of the distal portion, of the distal cap may be electropolished or electroplated. A distal front face of the distal cap may be entirely transparent.

In other aspects, a medical device may comprise a shaft extending to a distal portion, the distal portion of the shaft including: a side-facing imaging device; an electrical cable coupled to the side-facing imaging device; an illumination device positioned entirely proximal from the side-facing imaging device; and an optical fiber extending from the illumination device to a prism. The prism may be configured to redirect light from the illumination device towards a field of view of the side-facing imaging device.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. Although endoscopes are referenced herein, reference to endoscopes or endoscopy should not be construed as limiting the possible applications of the disclosed aspects. For example, the disclosed aspects may be used with duodenoscopes, bronchoscopes, ureteroscopes, colonoscopes, catheters, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of the this disclosure and together with the description, serve to explain the principles of the disclosure.

FIG. 1 is a perspective view of an endoscope system and a magnified view of the distal end of the endoscope of the endoscope system, according to aspects of this disclosure.

FIG. 2A is a perspective view of a distal tip section of a medical device, according to aspects of this disclosure.

FIG. 2B is a side view of the distal tip section of FIG. 2A, according to aspects of this disclosure.

FIGS. 3A and 3B show different perspective views of a distal tip section of a medical device, according to aspects of this disclosure.

FIG. 4 shows a top view of a distal tip section of a medical device, according to aspects of this disclosure.

FIG. 5 shows a top view of a distal tip section of a medical device, according to aspects of this disclosure.

FIG. 6 shows a top view of a distal tip section of a medical device, according to aspects of this disclosure.

FIG. 7 shows a perspective view of a distal tip section of a medical device, according to aspects of this disclosure.

FIG. 8 shows a perspective view of a distal tip section of a medical device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

Embodiments of this disclosure seek to improve lighting configurations for medical devices, and/or increase the available space for other components within medical devices. Embodiments of this disclosure seek to improve a physician's ability to visualize a target area and/or operate a medical device, such as an endoscope.

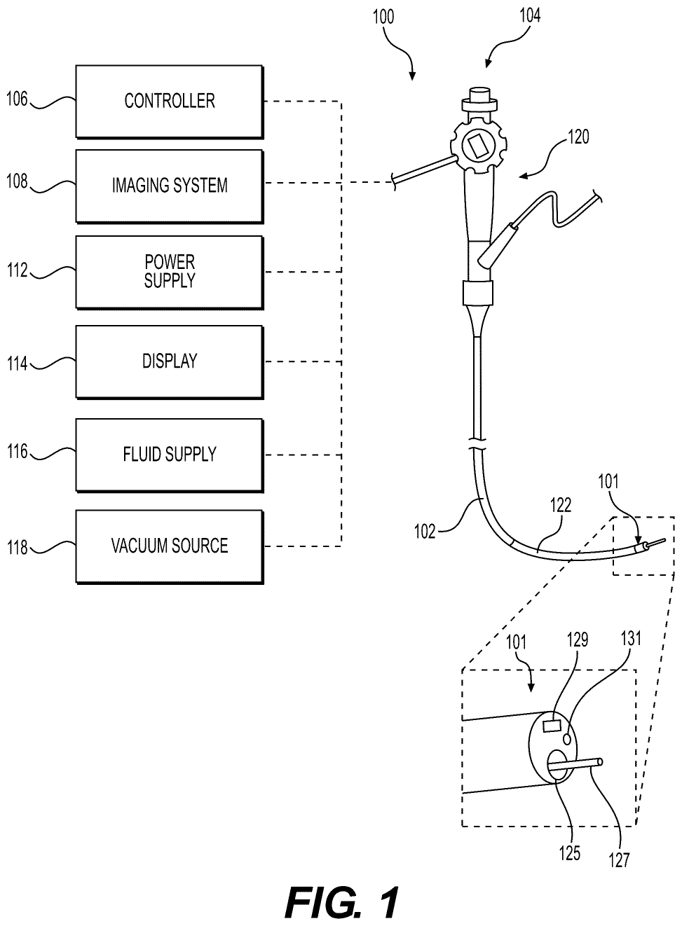

An exemplary endoscopy system 100 is shown in FIG. 1. Endoscopy system 100 may include an endoscope 104. Endoscope 104 may include a handle assembly 120 and a flexible tubular shaft 102. The flexibility of shaft 102 may be sufficient to allow shaft 102 to bend, to facilitate navigation of shaft 102 through a subject's tortuous anatomical passages. Shaft 102 may terminate at a distal tip 101. Shaft 102 may include an articulation section 122 for deflecting distal tip 101 in up, down, left, and/or right directions. In one example, articulation section 122 may provide for full retroflexion (e.g., rotation of distal tip 101 through an arc of 180 degrees) or only partial retroflexion (e.g., rotation of distal tip 101 through an arc of less than 180 degrees). Endoscope 104 also may include one or more lumens extending therethrough, and one or more openings in communication with the one or more lumens. For example, the one or more lumens may extend through handle assembly 120 and shaft 102, and the one or more openings may be on handle assembly 120 and distal tip 101. Endoscope 104 may be any suitable member for insertion into a patient's body, such as, e.g., an endoscope, a gastroscope, an ureteroscope, a nephroscope, a colonoscope, a hysteroscope, a ureteroscope, a bronchoscope, a cystoscope, a duodenoscope, a sheath, or a catheter.

One or more auxiliary devices may be operatively coupled to endoscope 104. Exemplary auxiliary devices may include a controller 106, an imaging system 108, a power supply 112, a display 114, a fluid supply 116, and/or a vacuum source 118, each of which is briefly described below. Controller 106 may include, for example, any electronic device capable of receiving, storing, processing, generating, and/or transmitting data according to instructions given by one or more programs. Controller 106 may be operatively coupled to, or part of, one or more of endoscope 104 and the other auxiliary devices, to control one or more aspects of their operation. Power supply 112 may include any suitable power source, and associated connectors (e.g., electrically-conductive wires), for supplying electronic components in the auxiliary devices and endoscope 104 with electrical power. Fluid supply 116 may include a reservoir, a medical irrigation bag, a pump, and any suitable connectors (e.g., tubing for fluidly coupling fluid supply 116 and endoscope 104). The pump may supply a flow of pressurized fluid to one or more of the lumens in endoscope 104, and the pressurized fluid flow may be emitted from distal tip 101 and/or used to inflate expandable components present at distal tip 101. Vacuum source 118 may provide suction or vacuum pressure to one or more lumens of the endoscope, and thereby provide a suction force or negative pressure to draw material toward and/or into endoscope 104, and/or to deflate expandable components.

Imaging system 108 may include imaging electronics to, for example, process signals received from an image sensor in endoscope 104, send signals for controlling the image sensor, adjust illumination levels of areas being viewed by the image sensor, and/or facilitate the display of image sensor data on display 114.

Distal tip 101 may include one or more image sensors 129 and one or more illuminators 131, shown in the magnified view of distal tip 101 in FIG. 1. One or more image sensors 129 may include a charge-coupled device image sensor, a complementary metal-oxide image semiconductor, or the like coupled to a cable or wire running through the shaft 102 of endoscope 104. One or more illuminators 131 may include light emitting diodes (LEDs) or the like. In some examples, one or more windows (not shown) may be in front of the one or more illuminators 131.

A tool 127 may be inserted into a working channel or lumen 125 of endoscope 104, and tool 127 may exit out of the distal end of lumen 125. Tool 127 may include, for example, a brush, such as a wire brush, a guidewire, cutting or grasping forceps, a biopsy device, a snare loop, an injection needle, a cutting blade, an electrosurgical knife, scissors, a retractable basket, a retrieval device, an ablation and/or electrophysiology catheter, a stent placement device, a surgical stapling device, a balloon catheter, a laser-emitting device, and/or any other suitable therapeutic or diagnostic accessory device. As shown in the magnified view of distal tip 101, tool 127 has a smaller circumference about its longitudinal axis compared to the circumference about the longitudinal axis of lumen 125, and may include a smaller cross-sectional diameter as compared to the diameter of lumen 125. Aspects of this disclosure provide embodiments of medical devices that increase the available space for components and/or tools 127 within distal tip 101. Additionally or alternatively, aspects of this disclosure may facilitate navigation through tortuous body pathways by increasing the amount of illumination within the user's field of view.

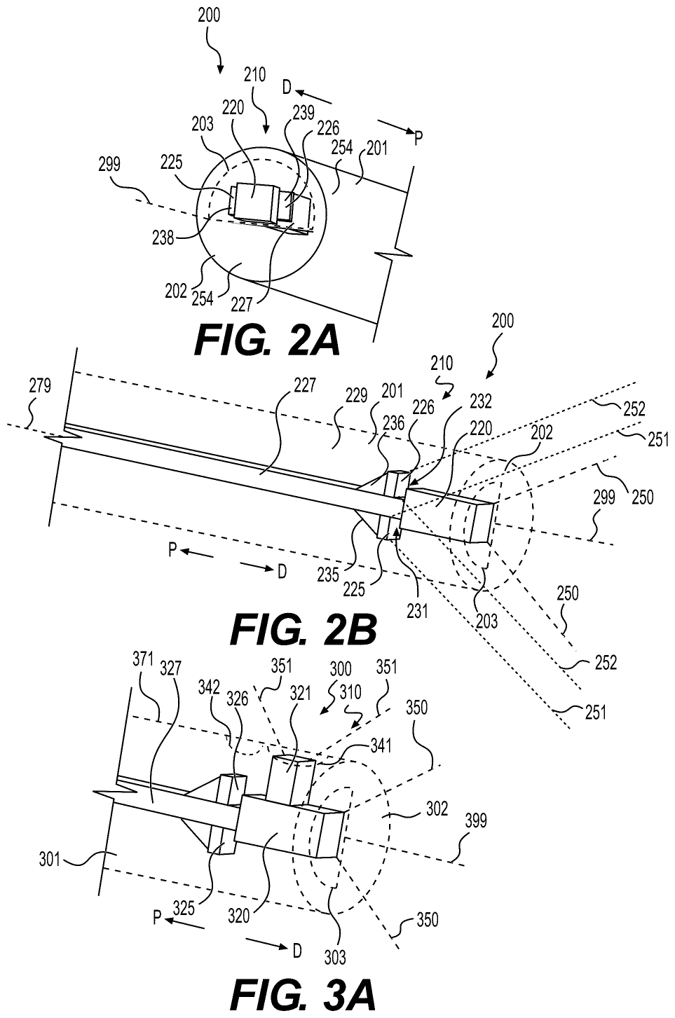

FIGS. 2A and 2B illustrate an exemplary distal portion of a medical device 200, with the shaft 201 of the medical device shown as transparent for illustration purposes to expose exemplary interior components of the medical device 200. Medical device 200 may include a shaft 201 terminating at a distal portion 210 including a distal front face 202 of the shaft 201. Medical device 200 may further include an imaging device 220, such as a camera or other optical assembly known in the art, for visualizing target anatomy at the distal portion 210 of medical device 200. In some examples, medical device 200 may include a window 203 on the distal front face 202 of shaft 201. An electrical cable 227 may be coupled to imaging device 220 and may extend proximally from imaging device 220 to a proximal portion of shaft 201. Two illumination devices 225, 226 may be positioned entirely proximal from imaging device 220 within distal portion 210. Illumination devices 225, 226 may be a light emitting diode device(s) (LEDs), fiber optic device(s), incandescent lamp(s) device, halogen light device(s), and/or any other type of light source known in the art. Although not shown, shaft 201 may include a lumen or working channel extending from the proximal end to distal front face 202.

Illumination devices 225, 226 may be directly or indirectly coupled to cable 227. As shown in FIG. 2B, illumination devices 225, 226 may be coupled to cable 227 via one or more substrates 235, 236. Substrates 235, 236 may be ceramic substrates, may be one or more adhesives, may be one or more electrical cables, and/or may be integrally formed with cable 227. Substrates 235, 236 may be triangular, as shown in FIG. 2B, rectangular, or any other suitable shape. In some examples, each illumination device 225, 226 may extend radially outward from a central longitudinal axis 279 of cable 227, and each field of illumination 251, 252 of illumination device 225, 226, respectively, may be facing a distal direction.

As shown in FIG. 2B, each field of illumination 251, 252 may cover the entirety of window 203 at distal front face 202. In other examples, each field of illumination 251, 252 may only partially cover window 203. Alternatively, one of the fields of illumination 251, 252 may entirely or partially cover window 203, and the other field of illumination 251, 251 may not cover any part of window 203. In some examples, as shown in FIG. 2B, imaging device 220 may be within each field of illumination 251, 252. In some examples, distal front face 202 may be positioned within a field of illumination 251, 252 of one of illumination devices 225, 226. In other examples, distal front face 202 may be at least partially within both fields of illumination 251, 252. Spaces 231, 232 are respectively provided between each illumination device 225, 226 and imaging device 220, which may help to reduce the total exposure of heat to imaging device 220 from illumination devices 225, 226.

By coupling illumination devices 225, 226 to cable 227 at a position entirely distal from imaging device 220, an improved thermal connection between illumination devices 225, 226 and cable 227 may be achieved, which may facilitate reducing the overall temperature of distal portion 210 during operation of medical device 200. Also, by positioning illumination devices 225, 226 within a central portion of distal portion 210 and spaced from an exterior surface 254 of distal portion 210, the temperature of the exterior surface 254 of distal portion 210 may be reduced during operation of medical device 200. For example, by not positioning illumination devices 225, 226 at or close to the exterior surface 254 of distal portion 210 and instead positioning illumination devices 225, 226 within a central portion of the interior of distal portion 210, the temperature at the exterior surface 254 of distal portion 210 may be reduced during operation of medical device 200. As shown in FIG. 2A, each distal front face 238, 239 of each illumination device 225, 226, respectively, may extend radially outward, relative to central longitudinal axis 279 of cable 227, radially beyond imaging device 220, thus providing a portion of each distal front face 238, 239 with an unobstructed path to window 203.

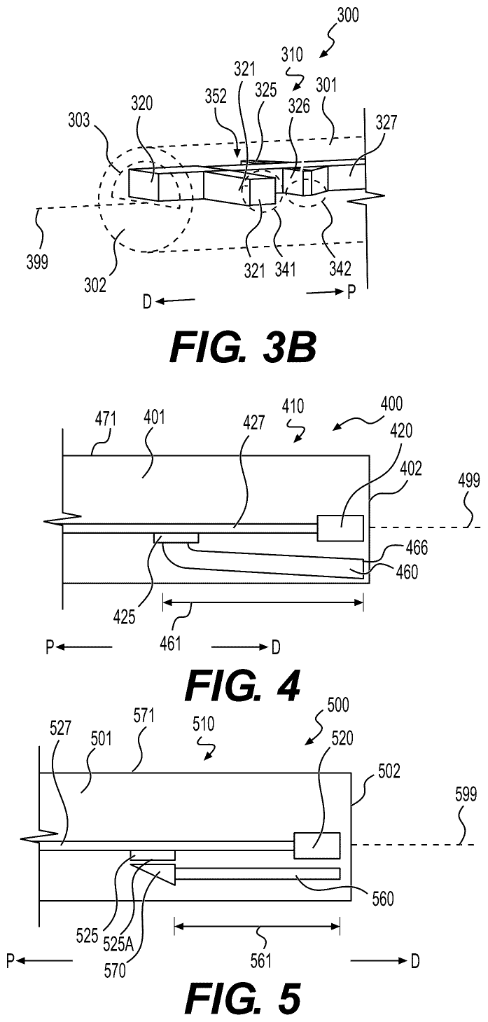

Window 203 may include transparent glass, transparent plastic, semitransparent material, and/or any other transparent material configured to allow light to pass through the material. Window 203 may be semi-circular (as shown in FIG. 2B), circular, rectangular, elliptically shaped or oval shaped, triangular, and/or any other suitable shape. In some examples, window 203 may be a portion of distal front face 202, or may be the entirety of distal front face 202. As will be discussed further in relation to FIGS. 3A-3B below, one or more windows 203 may be positioned at distal front face 202 and/or at one or more sidewalls 229 of distal portion 210. Window 203 may be aligned (laterally, longitudinally, etc.) with imaging device 220 and illumination devices 225, 226.

FIGS. 3A and 3B illustrate a distal portion 310 of an alternative embodiment of a medical device 300. Medical device 300 may have any of the features described hereinabove in relation to medical device 200. A shaft 301 of the medical device 300 is shown as transparent for illustration purposes to expose exemplary interior components of the medical device 300. Shaft 301 terminates at a distal portion 310 including a distal front face 302. Medical device 300 may further include a first imaging device 320 and a second imaging device 321. In some examples, medical device 300 may include a first window 303 on the distal front face 302 of shaft 301, and a second window 341 on a sidewall 371 of distal portion 310. An electrical cable 327 may be coupled to the first imaging device 320 and the second imaging device 321, and may extend proximally to a proximal portion of shaft 301. Two illumination devices 325, 326 may be positioned entirely proximal from both the first imaging device 320 and the second imaging device 321, and may be within distal portion 310.

As shown in FIG. 3A, first imaging device 320 may be a front-facing imaging device, and second imaging device 321 may be a side-facing imaging device. For example, a first field of view 350 of the first imaging device 320 may be facing distally, and a second field of view 351 of second imaging device 321 may be facing a direction transverse from a central longitudinal axis 399 of distal portion 310. First imaging device 320 may be aligned with first window 303, and second imaging device 321 may be aligned with second window 341. The first field of view 350 of the first imaging device 320 may extend through first window 303, and the second field of view 351 of the second imaging device 321 may extend through the second window 341.

Illumination devices 325, 326 may be configured to provide illumination to the first field of view 350 and the second field of view 351. For example, the field of illumination of illumination device 326 may overlap with both the first field of view 350 and the second field of view 351. In some examples, as shown in FIGS. 3A and 3B, an additional side window 342 may be positioned on sidewall 371 of distal portion 310 to allow additional illumination from illumination devices 325, 326 to reach the second field of view 351. Illumination devices 325, 326 may be positioned entirely proximal from both the first imaging device 320 and the second imaging device 321. Illumination devices 325, 326 may be spaced from sidewall 371 of distal portion 310, which may facilitate reducing the operating temperature of distal portion 310, for example reducing the surface temperature of sidewall 371 during operation of medical device 300, compared to configurations with illumination devices at sidewall 371.

FIG. 4 illustrates another embodiment of a medical device 400, including a distal portion 410, an imaging device 420, an electrical cable 427, a distal front face 402, and an illumination device 425. Medical device 400 may have any of the features described herein in relation to any of the other medical systems/devices 100, 200, 300. Although not depicted in FIG. 4, medical device 400 may include one or more windows, and in some examples, may have a distal cap (e.g., the entirety of distal front face 402 and a portion of sidewall 471 that is entirely transparent or partially transparent). Illumination device 425 may be directly coupled to electrical cable 427.

A field of illumination of illumination device 425 may face a direction transverse from central longitudinal axis 499, and an optical fiber 460 may be coupled to or positioned adjacent to illumination device 425. Optical fiber 460 may be a glass fiber, a glass fiber bundle, a plastic optical fiber, and/or any other optical fiber known in the art. Optical fiber 460 may have any suitable shape, and may be designed to fit between other components present within distal portion 410 of medical device 400. Optical fiber 460 may allow illumination device 425 to be mounted a larger distance (shown by distance marker 461 in FIG. 4) from imaging device 420 compared to systems or devices not utilizing an optical fiber, while still providing illumination to the field of view of imaging device 420. By allowing illumination device 425 to be mounted a large distance from imaging device 420, the temperature of distal portion 410 may be reduced by spacing apart components that produce a significant amount of heat. In some examples, illumination device 425 may be positioned 1 cm, 2 cm, 3 cm, 4 cm, 5 cm, or any other distance from imaging device 420. In some examples, optical fiber 460 may be replaced with an acrylic molded light guide, and, in some examples, distal portion 410 may include a distal cap made entirely of an acrylic molded light guide.

As shown in FIG. 4, optical fiber 460 may extend from illumination device 425 and curve approximately ninety degrees, and then extend longitudinal to a position proximate to distal front face 402. Optical fiber 460 may be configured to allow illumination device 425 to be placed orthogonal to the direction of a field of view of imaging device 420, and the orientation of illumination device 425 may reduce space occupied by illumination device 425 within the distal portion 410. A distal end 466 of optical fiber 460 may be facing the same direction that the field of view of imaging device 420 faces. In other examples (not shown), multiple optical fibers may be included in distal portion 410, with each optical fiber coupled to one or more illumination devices. In some examples, two optical fibers may be coupled to a single illumination device and may be oriented towards different directions. In other examples, two illumination devices may be coupled to electrical cable 427 and each illumination device may have an optical fiber coupled thereto. Optical fiber 460 may be used to adjust an emission angle of the illumination provided by illumination device 425. For example, angling distal end 466 of optical fiber 460 relative to distal front face 402 allows optical fiber 460 to direct light (or a center of the field of illumination of optical fiber 460) towards a center of a field of view of imaging device 420 or any other desired direction. In some examples, optical fiber 460 may be used to steer or direct illumination from illumination device 425 around imaging device 420, for example, to help prevent light from being directed onto the back (e.g., proximal end) of optical device 420.

FIG. 5 illustrates another embodiment of a medical device 500 including a distal portion 510, an imaging device 520, an electrical cable 527, a distal front face 502, and an illumination device 525. Medical device 500 may have any of the features described herein in relation to any of the other medical systems/devices 100, 200, 300, 400, 600, 700, 800. Although not depicted in FIG. 5, medical device 500 may include one or more windows, and in some examples may have a distal cap (e.g., the entirety of distal front face 502 and a portion of sidewall 571 that is entirely transparent or partially transparent). Illumination device 525 may be directly coupled to electrical cable 527. A field of illumination of illumination device 525 may face a direction transverse from central longitudinal axis 599, and a prism 570 may be coupled to and/or positioned adjacent to illumination device 525. Prism 570 may redirect the field of illumination of illumination device 525 by approximately ninety degrees, or redirect the light emitted from a light emitting surface of illumination device 525 to a direction approximately orthogonal to the light emitting surface 525A of illumination device 525 (shown in FIG. 5).

Although prism 570 is shown in FIG. 5 redirecting the light from illumination device 525 by ninety degrees, other prisms may replace prism 570 that redirect the light at different angles, such as by 30 degrees, 45 degrees, 60 degrees, 120 degrees, or any other suitable degree of redirection. As shown in FIG. 5, an optical fiber 570 may be coupled to or positioned adjacent to prism 570 to transfer the redirected light from prism 570 to a distal portion of medical device 500, such as to a position proximate to distal front face 502 (shown in FIG. 5). By incorporating prism 570, optical fiber 560 does not need to include a bent or curved portion, which may help reduce the tendency of optical fiber 560 breaking. In some examples, medical device 500 may not include an optical fiber 560, and prism 570 may redirect the light from illumination device 525 to a distal direction without the aid of optical fiber 560. In other examples, multiple prisms 570 may be used in distal portion 510 to redirect light from two different illumination devices, for example if two illumination devices 525 were coupled to electrical cable 527.

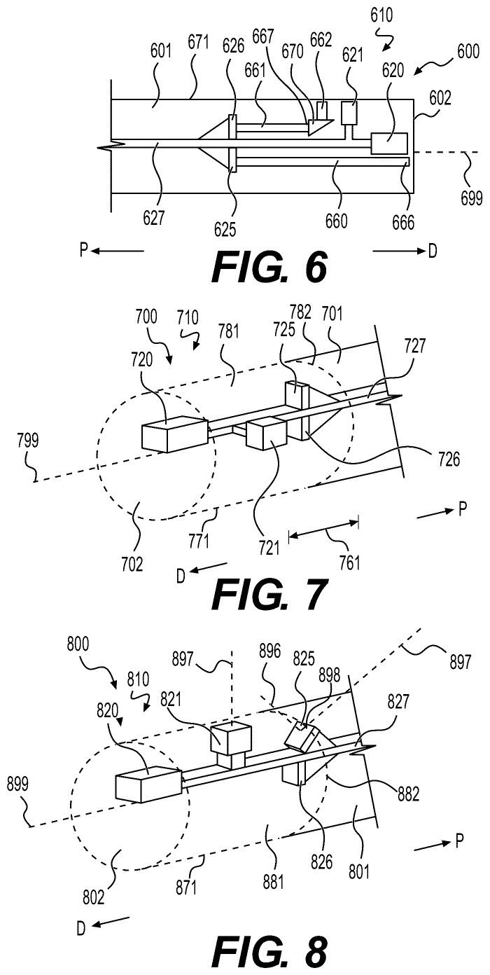

FIG. 6 illustrates another embodiment of a medical device 600 including a distal portion 610, a first imaging device 620, a second imaging device 621, an electrical cable 627, a distal front face 602, a first illumination device 625, and a second illumination device 626. Medical device 600 may have any of the features described herein in relation to any of the other medical systems/devices 100, 200, 300, 400, 500, 700, 800. Although not depicted in FIG. 6, medical device 600 may include one or more windows, and in some examples may have a distal cap (e.g., the entirety of distal front face 602 and a portion of sidewall 671 that is entirely transparent or partially transparent). First illumination device 625 and second illumination device 626 may be directly coupled to electrical cable 627 in the same manner illumination devices 225, 226 are coupled to electrical cable 227 in FIG. 2B. A field of illumination of each illumination device 625, 626 may face a direction substantially parallel to central longitudinal axis 699. A first optical fiber 660 may be coupled to first illumination device 625, may extend distally to a distal end 666 positioned proximate to distal front face 602, and may be substantially parallel to central longitudinal axis 699. Distal end 666 of optical fiber 660 may be adjacent to first imaging device 620, and may be configured to provide illumination for the field of view of the first imaging device 620.

A second optical fiber 661 may be coupled to the second illumination device 626 and may extend distally within distal portion 610 to a prism 670. A distal end 667 of the second optical fiber 661 may be coupled to prism 670, and prism may redirect the light provided from the second optical fiber 661 by approximately ninety degrees towards sidewall 671. A third optical fiber 662 may be coupled to prism 670 and may transfer light from prism 670 to a position proximate to sidewall 671. In some examples, the third optical fiber 662 may not be included in distal portion 610. Prism 670, and in some examples the third optical fiber 662, may direct light towards the field of view of second imaging device 621. Prism 670 and third optical fiber 662 may be adjacent to second imaging device 621. The design of medical device 600 allows illumination devices 625, 626 to be positioned within distal portion 610 and spaced imaging devices 620, 621 while still providing illumination for the field of view of each imaging device 620, 621.

FIG. 7 illustrates another embodiment of medical device 700 including a distal portion 710, a first imaging device 720, a second imaging device 721, an electrical cable 727, a distal front face 702, a first illumination device 725, and a second illumination device 726. Medical device 700 may have any of the features described herein in relation to any of the other medical systems/devices 100, 200, 300, 400, 500, 600, 800. Medical device 700 includes a distal cap 781 shown in dashed-lines that includes distal front face 702 and a sidewall 771 extending proximally from distal front face 702. Distal cap 781 may be configured to contain or enclose first imaging device 720, second imaging device 721, a distal portion of electrical cable 727, first illumination device 725, and second illumination device 726. In some examples, the proximal end 782 of distal cap 781 may be axially aligned with first illumination device 725 and/or second illumination device 726 (as shown in FIG. 7).

Distal cap 781 may be entirely clear, entirely transparent, and/or partially transparent. Distal cap 781 may have minimal absorption in the visual spectrum of light, therefore allowing for the light from illumination devices 725, 726 to be emitted through distal cap 781 and to the target anatomy. In some examples, distal cap 781 may be at least partially metal and may include one or more radially-inward facing, relative to central longitudinal axis 799, surfaces that is electropolished or otherwise treated to create a surface finish approximating a mirror. By including an electropolised radially-inward facing surface of distal cap 781, nearly total reflection within distal cap 781 minimizes losses of illumination the distal cap 781 and more illumination is emitted or sent out of distal cap 781 at target anatomy. In some examples, distal cap 781 may be plastic or any other suitable material, and electroplating may be applied to one or more radially-inward facing surfaces of distal cap 781 to create a mirror finish within distal cap 781. In some examples, a radially-inward facing surface of sidewall 771 may be electropolished or electroplated such that the electropolish or electroplating extends entirely around central longitudinal axis 799, and distal front face 702 may be a transparent material, thus directing light via the electropolishing or electroplating towards the transparent distal front face 702. In some examples, distal cap 781 may be transparent and may contain phosphor configured to convert the light provided from illumination devices 725, 726 from one color to another color, for example, from blue light to white light. For example, distal cap 781 could be molded with a phosphor material.

FIG. 8 illustrates another embodiment of medical device 800 including a distal portion 810, a first imaging device 820, a second imaging device 821, an electrical cable 827, a distal front face 802, a first illumination device 825, and a second illumination device 826. Medical device 800 may have any of the features described herein in relation to any of the other medical systems/devices 100, 200, 300, 400, 500, 600, 700. Medical device 800 includes a distal cap 881 shown in dashed-lines that includes distal front face 802 and a sidewall 871 extending proximally from distal front face 802. Distal cap 881 may be configured to contain or enclose first imaging device 820, second imaging device 821, a distal portion of electrical cable 827, first illumination device 825, and second illumination device 826.

As shown in FIG. 8, first illumination device 825 may be directed towards a central viewing axis 897 of second imaging device 821. Central viewing axis 897 may be a center axis of the field of view of second imaging device 821. A center illumination axis 896 of first illumination device 825 may be a center axis of the field of illumination of first illumination device 825. Center illumination axis 896 may be transverses from a central longitudinal axis 899 of distal portion 810, and center illumination axis 896 may intersect with center viewing axis 897. First illumination device 825 may be angled relative to central longitudinal axis 899, such that a front illumination surface 898 extends proximally outward from electrical cable 827. An axis 897 extending outward and parallel to front illumination surface 898 is shown in FIG. 8 to illustrate the angle of front illumination surface 898 relative to central longitudinal axis 899. By angling illumination surface 898 of first illumination device 825, light from first illumination device 825 may be directed more towards the field of view of second imaging device 821 (e.g., towards the side of distal portion 810). Since distal cap 881 extends proximally to a proximal end 882 axially aligned with first illumination device 825, illumination from first illumination device 825 may travel through distal cap 881 towards the field of view of second imaging device 821 when distal cap 881 is transparent or partially transparent.

Second illumination device 826 may face distally and may extend radially outward from electrical cable 827 at a substantially right angle, which helps to direct illumination from second illumination device 826 towards the field of view of the first imaging device 820 (e.g., the front-facing imaging device).

Any of the medical devices 100, 200, 300, 400, 500, 600, 700, 800 described herein may incorporate white light LEDs into one or more of their illumination devices. In other examples, any of the medical devices 100, 200, 300, 400, 500, 600, 700, 800 described herein may incorporate blue light LEDs into one or more of their illumination devices, and may convert the blue light to white light using phosphor. As discussed above, the conversion may be added or may occur at different parts of the medical device and using different processes known in the art. For example, the conversion may occur directly on an LED of the medical device, or may be spatially removed from the LED and/or the illumination device. The conversion from blue light to white light, for example using phosphor, may occur before or after the light travels through one or more optical fiber and/or before or after the light travels through one or more prism. In some examples, one or more windows 203, 303, 341, 342 of a medical device 100, 200, 300, 400, 500, 600, 700, 800 may include phosphor to convert blue light to white light.

In any of the above-described embodiments, a medical device 100, 200, 300, 400, 500, 600, 700, 800 may include one or more components that are metallic, a polymer, machined, formed, stamped, insert molded, or any combination thereof. Any aspect(s) of any one of the above-described embodiments of medical devices 100, 200, 300, 400, 500, 600, 700, 800 may be incorporated into any of the other medical devices 100, 200, 300, 400, 500, 600, 700, 800 described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a shaft extending to a distal portion, the distal portion of the shaft including:
a first imaging device;
an electrical cable coupled to the first imaging device and extending proximally from the first imaging device; and
a first illumination device positioned entirely proximal from the first imaging device, wherein the first illumination device is (i) coupled to the electrical cable and (ii) configured to illuminate a first field of view of the first imaging device.

2. The medical device of claim 1, wherein the distal portion further includes a first window at a distal front face of the shaft, wherein the first window is longitudinally aligned with the first imaging device and the first illumination device.

3. The medical device of claim 1, further comprising a second illumination device positioned entirely proximal from the first imaging device, wherein the second illumination device is (i) coupled to the electrical cable and (ii) configured to illuminate the first field of view of the first imaging device.

4. The medical device of claim 1, wherein a distal front face of the distal portion is entirely transparent.

5. The medical device of claim 4, wherein at least one sidewall of the distal portion is transparent.

6. The medical device of claim 1, further comprising a second imaging device, wherein the first illumination device is entirely proximal from the second imaging device.

7. The medical device of claim 6, wherein a first field of view of the first imaging device faces a distal direction, wherein a second field of view of the second imaging device faces a direction transverse from a central longitudinal axis of the distal portion, and wherein the first illumination device is configured to illuminate both the first field of view and the second field of view.

8. The medical device of claim 7, wherein a sidewall of the distal portion includes a window aligned with the second imaging device.

9. The medical device of claim 1, wherein the distal portion further includes an optical fiber coupled to the first illumination device and extending distally to a distal front face of the distal portion.

10. The medical device of claim 1, wherein the distal portion further comprises at least one transparent portion comprising phosphor, wherein the at least one transparent portion is configured to convert blue light to white light.

11. The medical device of claim 1, wherein the distal portion further comprises a prism configured to redirect light received from the first illumination device towards a field of view of the first imaging device.

12. The medical device of claim 11, wherein the distal portion further comprises a first optical fiber coupled to the prism.

13. The medical device of claim 12, wherein the distal portion further comprises a second optical fiber coupling the prism to the first illumination device.

14. The medical device of claim 1, wherein the first illuminator includes a first illumination surface, wherein the first illumination surface extends transverse from a central longitudinal axis of the distal portion.

15. The medical device of claim 14, wherein the distal portion further comprises a second imaging device, wherein the field of illumination of the first illumination device is also directed towards a second field of view of the second imaging device.

16. A medical device comprising:
a shaft extending to a distal portion, the distal portion of the shaft including:
a front-facing imaging device;
a side-facing imaging device;
an electrical cable coupled to the front-facing imaging device and the side-facing imaging device; and
a first illumination device positioned entirely proximal from the front-facing imaging device and the side-facing imaging device, wherein the first illumination device is configured to illuminate a first field of view of the first imaging device and a second field of view of the second imaging device.

17. The medical device of claim 16, wherein the distal portion further includes a distal cap, wherein the distal cap contains the front-facing imaging device, the side-facing imaging device, and the first illumination device; and wherein the distal cap is at least partially transparent.

18. The medical device of claim 17, wherein a radially-inward facing surface, relative to a central longitudinal axis of the distal portion, of the distal cap is electropolished or electroplated.

19. The medical device of claim 18, wherein a distal front face of the distal cap is entirely transparent.

20. A medical device comprising:
a shaft extending to a distal portion, the distal portion of the shaft including:
a side-facing imaging device;
an electrical cable coupled to the side-facing imaging device;
an illumination device positioned entirely proximal from the side-facing imaging device; and
an optical fiber extending from the illumination device to a prism;
wherein the prism is configured to redirect light from the illumination device towards a field of view of the side-facing imaging device.
